(19) United States
(12) Patent Application Publication
(10) Pub. No.: US 2025/0089218 A1
(43) Pub. Date: Mar. 13, 2025

(54) PERSONAL CARE COMPOSITIONS INCLUDING ANTIPERSPIRANT, DEODORANT, AND ASTRINGENT COMPOSITIONS

(71) Applicant: ELEMENTIS SPECIALTIES, INC., East Windsor, NJ (US)

(72) Inventors: Rey ORDIALES, East Windsor, NJ (US); Kimberly BURCH, East Windsor, NJ (US); Victoria CARDARELLI, East Windsor, NJ (US); Barbara M. SCHMIDT, East Windsor, NJ (US)

(21) Appl. No.: 18/955,778

(22) Filed: Nov. 21, 2024

Related U.S. Application Data
(63) Continuation-in-part of application No. PCT/US2024/031470, filed on May 29, 2024.
Application No. PCT/US2024/031470 is a continuation of application No. 18/200,953, filed on May 23, 2023.

(30) Foreign Application Priority Data
Aug. 23, 2023 (GB) 2312153.6

(51) Int. Cl. H05K 7/20; H05K 1/02

(52) U.S. Cl. CPC H05K 7/205; H05K 1/0201

(57) ABSTRACT

A personal care composition comprising a personal care agent is described. The personal care agent includes a natural material, or a material derived/obtained from the natural material or an aluminum-free synthetic/chemical material equivalent natural material, and a suitable carrier. The personal care composition and the personal care agent are effective for reducing or preventing perspiration, eliminating or masking body odor, toning or reducing the effects of skin aging, improving appearance, cleansing, or general aesthetics.

### CROSS-REFERENCE TO RELATED APPLICATIONS

This application is a continuation-in-part application of to International Application No. PCT/US2024/031470, filed May 29, 2024, which claims priority to U.S. patent application Ser. No. 18/203,953, filed May 31, 2023, and United Kingdom Application No. GB 2312153.6, filed Aug. 8, 2023.

### FIELD OF THE INVENTION

The invention relates to personal care compositions, such as antiperspirant compositions, deodorant compositions, astringent compositions, and personal care compositions related thereto, including but not limited to, shampoo and bodywash compositions. The invention also relates to methods of reducing or preventing perspiration, masking or preventing body odor, skin toning or reducing the effects of skin aging, improving appearance, cleansing, and general aesthetics, as well as the use of certain natural or naturally obtainable ingredients, particularly phytochemicals, and/or aluminum-free synthetic/chemical functional equivalents, as personal care agents, such as antiperspirant, deodorant, and astringent agents.

### BACKGROUND

Personal care compositions, such as antiperspirant compositions, deodorant compositions, astringent compositions, and compositions related thereto, such as but not limited to, shampoo and body wash compositions, are known in the art.

Antiperspirant compositions are a well-known type of product in the field of personal care and are used to eliminate or reduce body odor, which is important for daily hygiene. Deodorant compositions are well-known in the field of personal care and are used to eliminate or mask unpleasant smells, such as body odor. Astringent compositions are well-known in the field of personal care and are used to tone or tighten skin to reduce the effects of skin aging. These compositions are available in a variety of forms for administration, such as solid sticks, roll-ons, wipes, aerosol sprays, pump sprays, gel formulations, lotion formulations, and the like.

The antiperspirant compositions generally contain an active ingredient, often referred to as an antiperspirant agent, which may narrow or block the ducts of sweat glands to reduce or prevent perspiration. The antiperspirant agent may also provide a deodorizing effect if it is able to mask odors or it may have antimicrobial activity.

Deodorant compositions generally contain an active ingredient, often referred to as a deodorant agent, which typically includes one or more of an antiseptic, anti-bacterial, anti-microbial, fragrance, perfume, antibiotic and/or fragrance component. Body odor is reduced by either masking, neutralizing or eliminating the odor. In addition, these deodorant agents may also function by eliminating the odor causing bacteria.

Astringent compositions generally contain an active ingredient, often referred to as an astringent agent, that restricts, lessens in size, shrinks, or constricts the number of bodily fluids that are released from the sweat ducts or bodily pores. Astringents provide a skin toning effect to reduce skin aging.

Antiperspirant agents, deodorant agents, astringent agents, as well as related personal care agents, such as but not limited to, shampoo and body wash agents, in common use, generally include aluminum or aluminum-zirconium salts, such as aluminum chlorohydrate or aluminum zirconium tetrachlorohydrex gly (AZG). For example, with respect to antiperspirants, these salts precipitate in humid conditions on the skin and interact with proteins in the skin to form macromolecules, which plug pores, to reduce or prevent sweating.

However, there is a need in the art to design and implement personal care compositions and agents wherein the aluminum or aluminum-zirconium salts are substituted or combined with aluminum-free materials, which are natural materials, naturally derived or obtained materials, or synthetic/chemical functional equivalents thereof. For example, the search for alternative antiperspirant agents or compounds with similar functionalities has been ongoing to match or improve the sweat reduction properties of aluminum or aluminum zirconium salts in known antiperspirant agents. Similarly, the search for alternative agents or compounds with similar functionalities to deodorant and astringent agents as well as shampoo and body wash agents, has been ongoing to match or improve the deodorizing and toning properties, or cleansing properties, of aluminum or aluminum zirconium salts in known deodorants, and astringents, and related personal care compositions.

### SUMMARY OF THE INVENTION

In a first aspect, the invention provides a personal care composition, including but not limited to an antiperspirant, a deodorant, or an astringent composition, including a personal care agent (an active agent), including but not limited to an antiperspirant, a deodorant, or an astringent agent, respectively, wherein the personal care agent includes a natural material, e.g., a phytochemical or a material derived/derivable or obtained/obtainable therefrom, or an aluminum-free synthetic/chemical material that is an equivalent or imparts substantially equivalent functional properties as the phytochemical or the material derived/obtained from the phytochemical, and a carrier. The phytochemical is selected from a flavonoid, an ester of gallic acid, an ellagic acid, and a mixture of two or more thereof.

In the first aspect, the personal care composition includes a personal care agent that comprises an aluminum-free material or a blend thereof. For example, an antiperspirant, a deodorant, or an astringent composition comprises an antiperspirant, deodorant, or astringent agent, respectively, wherein the antiperspirant, deodorant, or astringent agent comprises an aluminum-free material or a blend thereof.

In a second aspect, the invention provides a personal care composition, including but not limited to an antiperspirant, a deodorant, or an astringent composition including a personal care agent, including but not limited to an antiperspirant, a deodorant, or an astringent agent, respectively, wherein the personal care agent includes a natural material, e.g., natural extract, or a material derived/derivable or obtained/obtainable therefrom, or an aluminum-free synthetic/chemical synthetic/chemical material that is an equivalent or imparts substantially equivalent functional properties as the natural extract or the material derived or obtained from the natural extract; and a carrier. The natural extract is selected from cinnamon extract, black ginger extract, green tea extract, Emblica officinalis extract, and a mixture thereof, or a natural extract selected from an extract of Kaempferia species, a tea, pomegranate, sweet chestnut, camu camu, kakadu plum and Emblica officinalis, and a mixture thereof.

A third aspect of the invention relates to a method of producing a personal care effect selected from reducing or preventing perspiration, eliminating or masking body odor, toning or reducing the effects of skin aging, improving appearance, cleansing, or general aesthetics. The method comprises applying a personal care composition, in accordance with the first or second aspects of the invention, to skin of a human.

A fourth aspect of the invention relates to the use of a natural material, e.g., phytochemicals or plant (natural) extracts, as well as materials derived/derivable or obtained/obtainable therefrom or aluminum-free synthetic/chemical functional equivalents thereof, as a personal care agent for reducing or preventing perspiration, eliminating or masking body odor, toning or reducing the effects of skin aging, improving appearance, cleansing, or general aesthetics. In certain embodiments, the natural material is selected from an optionally substituted benzenepropenoic acid, a flavonoid, an ester of gallic acid, an ellagic acid or a mixture of two or more thereof or, preferably, is selected from a flavonoid, an ester of gallic acid, an ellagic acid or a mixture of two or more thereof. A natural extract is selected from cinnamon extract, black ginger extract, green tea extract, Emblica officinalis extract and a mixture of two or more thereof; preferably, the natural extract is selected from black ginger extract, green tea extract, Emblica officinalis extract and a mixture of two or more thereof. Additionally or alternatively, the natural extract is an extract of a Kaempferia species (e.g. black thai ginger, silver spot ginger, aromatic ginger, peacock ginger), a tea (e.g. green tea, black tea, oolong tea, white tea), pomegranate, sweet chestnut, camu camu, kakadu plum or Emblica officinalis.

A fifth aspect of the invention relates to a method of preparing a personal care composition including preparing a personal care agent by mixing a natural material, e.g., phytochemical or natural extract, or a material derived/derivable or obtained/obtainable therefrom, or an aluminum-free synthetic/chemical materials that demonstrate or impart equivalent or substantially equivalent functional properties as the phytochemical, the natural extract, or the material derived/obtained from the phytochemical of the natural extract; and a carrier. The phytochemical is selected from the group consisting of a flavonoid, an ester of gallic acid, ellagic acid or a mixture of two or more thereof, and the natural extract is selected from the group consisting of cinnamon extract, black ginger extract, green tea extract, Emblica officinalis extract, extract of a pomegranate, extract of a sweet chestnut, extract of a camu camu, extract of a kakadu plum, extract of black thai ginger, extract of silver spot ginger, extract of aromatic ginger, extract of peacock ginger, and a mixture thereof. Optionally, the personal care composition additionally includes one or more of a lubricant, fatty alcohol, starch and thickener. In certain embodiments, the lubricant, fatty alcohol, and carrier are combined to form a mixture; the mixture is heated; optionally, to a temperature effective to melt solids present, and a thickener is added, dispersing the thickener. A starch is then added, followed by addition of the a phytochemical or natural extract, or a material derived/derivable or obtained/obtainable therefrom, or an aluminum-free synthetic/chemical functional equivalent, as a personal care agent for reducing or preventing perspiration, eliminating or masking body odor, toning or reducing the effects of skin aging, improving appearance, cleansing, or general aesthetics, and then cooling the mixture.

In certain embodiments, the natural material is selected from black thai ginger, green tea extract, trans-cinnamic acid, Emblica officinalis extract and a mixture thereof, preferably the natural extract is selected from black ginger extract, green tea extract, Emblica officinalis extract and a mixture thereof. Further, the personal care agent is absent of aluminum or aluminum-zirconium salts. Additionally or alternatively, the natural extract is an extract of a Kaempferia species (e.g. black thai ginger, silver spot ginger, aromatic ginger, peacock ginger), a tea (e.g. green tea, black tea, oolong tea, white tea), pomegranate, sweet chestnut, camu camu, kakadu plum or Emblica officinalis. The phytochemical is selected from an optionally substituted benzenepropenoic acid, a flavonoid, an ester of gallic acid, an ellagic acid or a mixture of two or more thereof or, preferably, is selected from a flavonoid, an ester of gallic acid, an ellagic acid or a mixture thereof.

In certain embodiments, the personal care agent is a mixture or blend of about 1.5 wt. % black thai ginger and about 1.5 wt. % trans-cinnamic acid, or a mixture or blend of about 1.5 wt. % green tea extract and about 1.5 wt. % Emblica officinalis extract.

Optionally, the personal care composition further includes cyclomethicone, aluminum starch succinate, phenyl trimethicone, butyl ether, fumed silica, hydrogenated castor oil and stearyl alcohol.

### DETAILED DESCRIPTION OF PREFERRED EMBODIMENTS

The following definitions apply to the disclosure herein, including all aspects of the invention and their embodiments.

The term “personal care” as used herein, whether used alone or in conjunction with another term, refers to a non-therapeutic use, such as a non-therapeutic, cosmetic use. This term typically refers to use for personal hygiene. The term “personal care” includes leave-on, or rinse-off compositions and agents applied to the human body for improving appearance, cleansing, odor control, sweat control, toning, or general aesthetics. The personal care compositions and agents include the forms of a liquid, lotion, cream, foam, scrub, emulsion, and gel. Non-limiting examples include leave-on skin lotions and creams, shampoos, conditioners, shower gels, antiperspirants, deodorants, astringents, and the like. The term “skin” refers to skin on the face and body, such as, neck, chest, back, arms, underarms, hands, legs, buttocks, and scalp.

The compounds and intermediates described herein may be named according to the IUPAC (International Union for Pure and Applied Chemistry) or the CAS (Chemical Abstracts Service) nomenclature systems or by their commercial trade names. It should also be understood that any reference to substituted benzenepropanoic acid encompasses all stereoisomers (e.g. cis and trans isomers) of such compounds.

The term “phytochemical” as used herein refers to a chemical compound that is produced by a plant (e.g. through primary or secondary metabolism). In other words, a phytochemical is a naturally occurring, biologically active compound found in a plant. The phytochemical typically has biological activity that protects the plant from the environment, disease or a predator.

The term “flavonoid” as used herein refers to a compound having a 15-carbon core structure (e.g. skeleton) consisting of two phenyl rings (referred to as the “A” and “B” rings) and a heterocyclic ring (referred to as the “C” ring), which contains an oxygen atom. The term “flavonoid” is used in the conventional, restricted sense as referring only to those compounds with a C₆—C₃—C₆ carbon framework (e.g. core structure or skeleton) having the structure of a chromane or a 1-benzopyran (chromene), in which the fused benzene ring is designated as ring A and the 3,4-dihydro-2H-pyran or the pyran as ring C, along with a phenyl group (ring B) on ring C (see, for example, the “Nomenclature of flavonoids” by A. P. Rauter et al.—Reference 1). The general structure of a flavonoid with ring labelling is shown below.

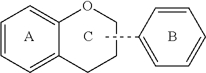

Depending on the position of the linkage of ring B to the chromane/1-benzopyran (chromene) moiety, the term “flavonoid” comprises, or consists of, the sub-classes of compounds known as 2-flavonoids, 3-flavonoids (e.g. also known as isoflavonoids) and 4-flavonoids (e.g. also known as neoflavonoids).

The term “2-flavonoid” as used herein refers to a compound having a core structure or skeleton as represented by formula (A-1).

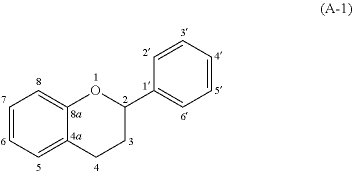 [SMILES: c1ccc(C2CCc3ccccc3O2)cc1]

In formula (A-1), the atoms of the core structure are numbered in accordance with the IUPAC numbering scheme for this sub-class of compounds.

For the avoidance of doubt, formula (A-1) represents the core structure or skeleton of a 2-flavonoid. Rings A to C of the core structure may include substituents. Ring C may include a carbon-carbon double bond (in addition to the unsaturation arising from the fused phenyl group, which is ring A) and/or a carbonyl group.

In general, the term “2-flavonoid” includes a flavan, a flavone or a flavanol.

When ring C in formula (A-1) does not include a carbon-carbon double bond or a carbonyl group, then formula (A-1) represents a flavan. The term “flavan” refers to a compound with a 2-phenyl-3,4-dihydro-2H-1-benzopyran skeleton.

When ring C in formula (A-1) is substituted with a hydroxy group, such as at the 3-position and/or the 4-position of ring C, then the 2-flavonoid is a flavanol.

The core structure or skeleton of the flavanol may be a flavan-3-ol (as represented by formula (A-2)), a flavan-4-ol (as represented by formula (A-3)) or a flavan-3,4-diol (as represented by formula (A-4)).

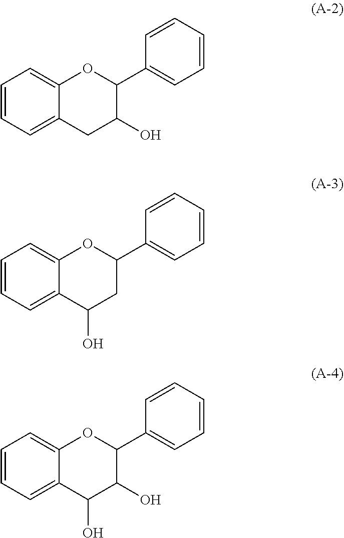 [SMILES: OC1Cc2ccccc2OC1c1ccccc1 ; OC1CC(c2ccccc2)Oc2ccccc21 ; OC1c2ccccc2OC(c2ccccc2)C1O]

When ring C in formula (A-1) includes a carbonyl group, such that the compound is derived from a 2-phenyl-2,3-dihydro-4H-1-benzopyran-4-one skeleton, then the 2-flavonoid is a flavanone, specifically a flavan-4-one (e.g. as represented by formula (A-5)).

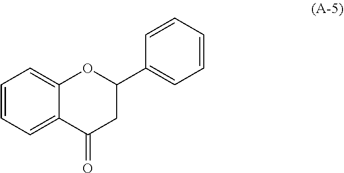 [SMILES: O=C1CC(c2ccccc2)Oc2ccccc21]

It is preferred that the core structure or skeleton of the 2-flavonoid is not an anthocyanidin (also known as an anthocyanin). An anthocyanidin is a compound derived from a flavylium (2-phenyl-1λ⁴-benzopyran-1-ylium or 2-phenylchromenylium) ion.

The term “3-flavonoid” or “isoflavonoid” as used herein refers to a compound having a core structure or skeleton as represented by formula (B-1).

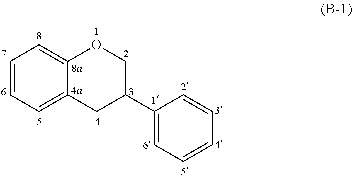 [SMILES: c1ccc(C2COc3ccccc3C2)cc1]

In formula (B-1), the atoms of the core structure are numbered in accordance with the IUPAC numbering scheme for this sub-class of compounds.

For the avoidance of doubt, formula (B-1) represents the core structure or skeleton of a 3-flavonoid. Rings A to C of the core structure may include substituents. Ring C may include a carbon-carbon double bond (in addition to the unsaturation arising from the fused phenyl group, which is ring A) and/or a carbonyl group.

In general, the term “3-flavonoid” or “isoflavonoid” includes isoflavans and isoflavones.

When ring C in formula (B-1) does not include a carbon-carbon double or a carbonyl group, then formula (B-1) represents an isoflavan. The term “isoflavan” refers to a compound with a 3-phenyl-3,4-dihydro-2H-1-benzopyran skeleton.

When ring C in formula (B-1) includes a carbonyl group, such as, for example, when the compound is derived from a 3-phenyl-4H-1-benzopyran-4-one (3-phenyl-4H-chromen-4-one) skeleton, then the 3-flavonoid is an isoflavanone (e.g. as represented by formula (B-2)).

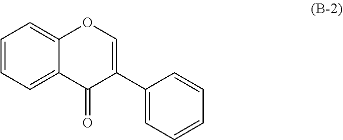 [SMILES: O=c1c(-c2ccccc2)coc2ccccc12]

The term “4-flavonoid” or “neoflavonoid” as used herein refers to a compound having a core structure or skeleton as represented by formula (C-1).

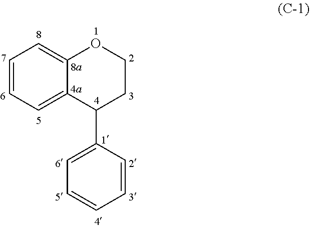 [SMILES: c1ccc(C2CCOc3ccccc32)cc1]

In formula (C-1), the atoms of the core structure have numbered in accordance with the IUPAC numbering scheme for this sub-class of compounds.

For the avoidance of doubt, formula (C-1) represents the core structure or skeleton of a 4-flavonoid. Rings A to C of the core structure may include substituents. Ring C may include a carbon-carbon double bond (in addition to the unsaturation arising from the fused phenyl group, which is ring A) and/or a carbonyl group.

In general, the term “4-flavonoid” or “neoflavonoid” includes neoflavans and neoflavones.

When ring C in formula (C-1) does not include a carbon-carbon double or a carbonyl group, then formula (C-1) represents a neoflavan. The term “neoflavan” refers to a compound with a 4-phenyl-3,4-dihydro-2H-1-benzopyran skeleton.

When ring C in formula (C-1) includes a carbonyl group, such as, for example, when the compound is derived from a 4-phenyl-2H-1-benzopyran-2-one (4-phenyl-2H-chromen-2-one) skeleton, then the 4-flavonoid is a neoflavanone (e.g. as represented by formula (C-2)).

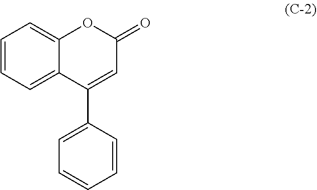 [SMILES: O=c1cc(-c2ccccc2)c2ccccc2o1]

The term “ellagic acid” as used herein refers to the following compound.

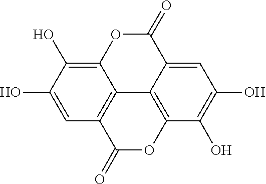 [SMILES: O=c1oc2c(O)c(O)cc3c(=O)oc4c(O)c(O)cc1c4c23]

Ellagic acid is a polyphenol, which is obtained or is obtainable from the natural extracts of many fruits, vegetables and nuts, such as blackberries, raspberries, strawberries, cranberries, walnuts, pecans, pomegranates, wolfberries, sweet chestnut, camu camu, and kakadu plum.

The term “glucogallin” as used herein refers to the following compound.

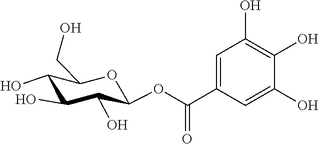

Glucogallin is typically present in a natural extract of Emblica officinalis. Thus, glucogallin is obtained or is obtainable from Emblica officinalis extract.

The term “black ginger” or “black thai ginger” as used herein refers to Kaempferia parviflora and Kaempferia angustifolia, preferably Kaempferia parviflora. The term “silver spot ginger” as used herein refers to Kaempferia elegans. The term “aromatic ginger” as used herein refers to Kaempferia galanga or Kaempferia larsenii. The term “peacock ginger” as used herein refers to Kaempferia rotunda.

The term “tea” as used herein refers to Camellia sinensis.

The term “pomegranate” as used herein refers to Punica granatum.

The term “sweet chestnut” as used herein refers to Castanea sativa.

The term “camu camu” as used herein refers to Myrciaria dubia.

The term “kakadu plum” as used herein refers to Terminalia ferdinandiana.

The term “saberry” or “saberry extract” as used herein is synonymous with Emblica officinalis or Emblica officinalis extract, respectively, and refers to fruits or an extract of fruits of Emblica officinalis (synonymous with phyllanthus emblica), which is more commonly known as amla berry or Indian gooseberry.

As used herein by itself or in conjunction with another term or terms, “substituted” indicates that a hydrogen atom on a molecule has been replaced with a different atom or group of atoms. The atom or group of atoms replacing the hydrogen atom is a “substituent.” It should be understood that the terms “substituent”, “substituents”, “moiety”, “moieties”, “group”, or “groups” refer to substituent(s).

The various hydrocarbon-containing moieties provided herein may be described using a prefix designating the minimum and maximum number of carbon atoms in the moiety, namely “Cₐ-C_{b}”. For example, Cₐ-C_{b} alkyl indicates an alkyl moiety having the integer “a” to the integer “b” number of carbon atoms, inclusive.

As used herein, the term “alkyl”, whether used alone or in conjunction with another term, refers to a branched or unbranched saturated hydrocarbon chain, unless specified otherwise. An alkyl group is typically unsubstituted, unless indicated otherwise. An alkyl group may have 1 to 6 carbon atoms, such as 1 to 4 carbon atoms. Representative examples include, but are not limited to, methyl, ethyl, n-propyl, i-propyl, n-butyl, i-butyl, s-butyl, t-butyl, n-pentyl, n-hexyl.

As used herein, the term “alkoxy” refers to an alkyl-oxy group, wherein the alkyl group is as previously defined. An alkoxy group is typically unsubstituted, unless specified otherwise. Representative examples include —OMe, —OEt, —OⁿPr, —OⁱPr.

The term “deodorant” as used herein refers to an agent, material or composition that masks or inhibits the formation of unpleasant body odors.

The term “natural extract” as used herein includes a product that is derived (or derivable) or obtained (or obtainable) from natural sources, such as plants, fruits, vegetables, botanicals, herbs, and spices. The process of making an extract involves combining raw materials with a mixture of water and ethanol while heating them. After heating, the solids and liquids are separated, and the solids are concentrated into an extract.

Amounts of a material, such as a compound, composition or an ingredient, are typically defined in terms of the wt % of the composition.

The term “about” when used herein in conjunction with a measurable value encompasses reasonable variations of the value, for instance, to allow for experimental error in the measurement of said value.

As used in the present disclosure, the term “comprises” has an open meaning, which allows other, unspecified features to be present. This term embraces, but is not limited to, the semi-closed term “consisting essentially of” and the closed term “consisting of”. Unless the context indicates otherwise, the term “comprises” may be replaced with either “consisting essentially of” or “consists of”. The term “consisting essentially of” may also be replaced with “consists of”.

For the avoidance of doubt, the expression “consists essentially of” as used herein limits the scope of a feature to include the specified materials, and any other materials or steps that do not materially affect the basic and novel characteristics of that feature, such as, for example, minor impurities. The expression “consists essentially of” embraces the expression “consisting of”.

### Personal Care Agent

The invention relates to the use of natural materials, sources or products or those that are derived/derivable or obtained/obtainable therefrom or aluminum-free synthetic/chemical materials that demonstrate or impart equivalent or substantially equivalent functional properties as the natural or derived/derivable or obtained/obtainable materials or products, as personal care compositions and agents.

It has surprisingly been found that certain classes of phytochemicals or plant (natural) extracts, as well as materials derived/derivable or obtained/obtainable therefrom or aluminum-free synthetic/chemical functional equivalents thereof, possess personal care properties, such as but not limited to, antiperspirant, deodorant, and astringent properties; therefore, they are useful and effective as personal care agents, such as but not limited to, antiperspirant, deodorant, and astringent agents, particularly, in personal care compositions. These materials provide a personal care effect, e.g., an antiperspirant, a deodorant, or an astringent effect, comparable to the aluminum or aluminum-zirconium salts that are typically included in most commercially available personal care compositions, e.g., antiperspirant, deodorant, and astringent compositions, respectively. Thus, the phytochemicals or plant (natural) extracts, materials derived/derivable or obtained/obtainable therefrom, or aluminum-free synthetic/chemical functionally equivalents are included as personal care agents, e.g., antiperspirant, deodorant, or astringent agents, instead of or in combination with the aluminum or aluminum-zirconium salts. These materials, particularly the natural extracts and those derived/derivable therefrom, may also impart a pleasant odor to the personal care composition.

The personal care compositions and agents are for providing a personal care effect. The personal care compositions and agents are for reducing or preventing perspiration, eliminating or masking body odor, toning or reducing the effects of skin aging, improving appearance, cleansing, or general aesthetics. In certain embodiments, an antiperspirant agent is an active ingredient for reducing or preventing perspiration in an antiperspirant composition of the invention, a deodorant agent is an active ingredient for eliminating or masking body odor in a deodorant composition of the invention, and an astringent agent is an active ingredient for toning or reducing the effect of skin aging in an astringent composition of the invention.

Perspiration is a physiological body function that helps to control and preserve body temperature at approximately 37° C. Sweat is produced by sweat glands, which are cutaneous annexes composed by an outer layer of myoepithelial contractible cells and an inner layer of secreting cells. When the body temperature increases, the sweat glands are activated by a signal sent by the brain to produce sweat. Besides preserving stability of body temperature, sweat also humidifies skin and releases toxins. Excess transpiration can cause discomfort, such as underarm bad odor and lesions.

In certain embodiments, the personal care composition, e.g., antiperspirant, works by limiting the quantity of sweat released by the skin through the ducts of sweat glands by forming a buffer.

The personal care agent of the invention is for external use (e.g. on the human body).

The personal care composition or the personal care agent is for application to a surface of a body, particularly the skin.

The personal care agent includes a flavonoid, an ester of gallic acid, ellagic acid or a mixture of two or more thereof. Optionally, the personal care agent consists essentially of a flavonoid, an ester of gallic acid, an ellagic acid or a mixture of two or more thereof.

Flavonoids are a class of polyphenolic compounds, which are typically secondary metabolites found in plants.

In certain embodiments, the personal care agent includes a natural material selected from black thai ginger, green tea extract, trans-cinnamic acid, Emblica officinalis extract, and mixtures or blends thereof, and is absent of aluminum or aluminum-zirconium salts. Optionally, the personal care agent consists of black thai ginger or green tea extract. Optionally, each of the black thai ginger or the green tree extract is present in an amount of 3 wt. %. Optionally, the personal care agent consists of black thai ginger and trans-cinnamic acid. Optionally, each of the black thai ginger and the trans-cinnamic acid is present in an amount of 1.5 wt. % (for a total of 3.0 wt. % based on total weight of the personal care composition). Optionally, the personal care agent consists of green tea extract and Emblica officinalis extract. Optionally, each of the green tea extract and Emblica officinalis extract is present in an amount of 1.5 wt. % (for a total of 3.0 wt. % based on total weight of the personal care composition).

In addition to the personal care agent, the personal care composition comprises cyclomethicone, aluminum starch succinate, phenyl trimethicone, butyl ether, fumed silica, hydrogenated castor oil and stearyl alcohol.

The personal care agent comprises a phytochemical selected from a flavonoid, an ester of gallic acid, an ellagic acid or a mixture of two or more thereof, or a material derived/obtained from the phytochemical, or an aluminum-free synthetic/chemical material that is an equivalent or imparts substantially equivalent functional properties as the phytochemical or the material derived/obtained from the phytochemical. In certain embodiments, the personal care agent includes ellagic acid. Optionally, the personal care agent includes an ester of gallic acid. In certain embodiments, the ester of gallic acid is selected from a flavonoid ester of gallic acid and a sugar ester of gallic acid. Optionally, the ester of gallic acid is a sugar ester of gallic acid. Optionally, the sugar ester of gallic acid is glucogallin, such as, β-glucogallin. Optionally, the ester of gallic acid is a flavonoid ester of gallic acid. Optionally, the flavonoid ester of gallic acid is a flavan-3-ol ester of gallic acid.

The flavan-3-ol ester of gallic acid includes the following structural moiety:

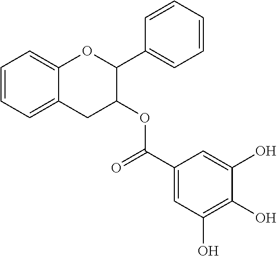 [SMILES: O=C(OC1Cc2ccccc2OC1c1ccccc1)c1cc(O)c(O)c(O)c1]

The term “moiety” is used because the above structure may contain one or more substituents on the 2-flavonoid skeleton or core structure.

In certain embodiments, the flavan-3-ol ester of gallic acid is selected from catechin gallate, epicatechin gallate, epigallocatechin gallate and a mixture of two or more thereof. Optionally, the flavan-3-ol ester of gallic acid is epigallocatechin gallate. In certain embodiments, a green tea extract contains at least 30 wt % of epigallocatechin gallate. Optionally, the flavan-3-ol ester of gallic acid is selected from catechin gallate, epicatechin gallate and a mixture thereof. In certain embodiments, a cinnamon extract contains a mixture of catechin gallate and epicatechin gallate. Optionally, the personal care agent is a combination or a mixture of the ester of gallic acid and ellagic acid.

In certain embodiments, the personal care agent includes a flavonoid. The flavonoid is a 2-flavonoid, a 3-flavonoid or a 4-flavonoid. The flavonoid has the general structure:

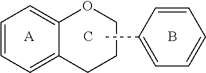

Optionally, the flavonoid is a 2-flavonoid, a 3-flavonoid or a mixture of two or more thereof. Optionally, the flavonoid is a 2-flavonoid or a mixture of 2-flavonoids. In certain embodiments, the flavonoid has at least one substituent on ring A, and wherein each substituent is independently selected from hydroxy (—OH) and C₁₋₆ alkoxy. Optionally, the flavonoid has two or more substituents on ring A, wherein each substituent is independently selected from hydroxy and C₁₋₆ alkoxy. Optionally, the C₁-C₆ alkoxy is either ethoxy or methoxy. Optionally, the C₁-C₆ alkoxy is methoxy. Optionally, each substituent is hydroxy. Optionally, the two or more substituents on ring A, are hydroxy (—OH). Optionally, one substituent on ring A is at the 5-position. Optionally, one substituent on ring A is at the 7-position. Optionally, the two or more substituents on ring A, are at least at the 5-position and the 7-position of ring A.

Optionally, each flavonoid has at least one substituent on ring B, wherein each substituent is independently selected from hydroxy (—OH) and C₁₋₆ alkoxy. Optionally, the flavonoid has two or more substituents on ring B, wherein each substituent is independently selected from hydroxy and C₁₋₆ alkoxy. Optionally, the flavonoid has three or more substituents on ring B, wherein each substituent is independently selected from hydroxy and C₁₋₆ alkoxy. Optionally, the C₁-C₆ alkoxy is either ethoxy or methoxy. Optionally, the C₁-C₆ alkoxy is methoxy. Optionally, each substituent is hydroxy. Optionally, the at least one substituent on ring B is hydroxy (—OH). Optionally, the two or more substituents on ring B are hydroxy (—OH). Optionally, the three or more substituents on ring B are hydroxy (—OH). Optionally, at least one substituent is at the 3′-position, the 4′-position or the 5′position of ring B, preferably the 4′-position of ring B. Optionally, the two or more substituents are at the 4′-position and the 3′-position of ring B. Optionally, the three or more substituents are at the 3′-position, the 4′-position and the 5-position of ring B.

In certain embodiments, the 2-flavonoid is a flavanol or an ester of a flavanol, or the mixture of 2-flavonoids includes a flavanol and/or an ester of a flavanol. Optionally, each flavanol is a flavan-3-ol. Optionally, the 2-flavonoid is afzelechin, epiafzelechin, catechin, epicatechin, gallocatechin, epigallocatechin or a combination of two or more thereof, optionally, the 2-flavonoid is catechin. In certain embodiments, a green tea extract contains at least 40 to 70 wt % of catechin. Optionally, the 2-flavonoid is a combination of catechin, epicatechin, gallocatechin and epigallocatechin. In certain embodiments, a cinnamon extract contains catechin, epicatechin, gallocatechin and epigallocatechin as its main ingredients. Optionally, the ester of a flavanol is an ester of a flavan-3-ol. Optionally, the ester of a flavan-3-ol is not a flavan-4-one, e.g., the flavan-3-ol does not have a carbonyl group at the 4-position on ring C. Optionally, the ester of a flavan-3-ol is a gallate ester of a flavan-3-ol, such as catechin gallate, epicatechin gallate, epigallocatechin gallate. Optionally, the 2-flavonoid is a flavanone, or the mixture of 2-flavonoids includes a flavanone. Optionally, the flavanone is a flavan-4-one. In certain embodiments, the flavan-4-one has at least one substituent at the 5-position or the 7-position of ring A, wherein the at least one substituent is C₁₋₆ alkoxy. Optionally, the C₁-C₆ alkoxy is either ethoxy or methoxy. Optionally, the C₁-C₆ alkoxy is methoxy.

In certain embodiments, the flavan-4-one is selected from rutin, 5,7-dimethoxyflavan-4-one, 5-hydroxy-7-methoxyflavan-4-one and a mixture of two or more thereof. Optionally, the flavan-4-one is rutin. Optionally, the cinnamon extract contains rutin.

In certain embodiments, the flavan-4-one is 5,7-dimethoxyflavan-4-one. In certain embodiments, the black ginger extract contains 5,7-dimethoxyflavan-4-one.

Flavonoids are often coloured, whereas the optionally substituted benzenepropenoic acid is a white solid.

In certain embodiments, the personal care agent is a combination or a mixture of the flavonoid and the ester of gallic acid. Optionally, the personal care agent is a combination or a mixture of the flavonoid and the gallic acid.

In certain embodiments, the personal care agent includes catechin and epigallocatechin gallate. Optionally, catechin is present in an amount of at least 40 wt. % or 75 wt. % (e.g., based on total wt. of the personal care agent).

In certain embodiments, the personal care agent includes at least 15 wt. % epigallocatechin gallate (e.g., based on total wt. % of the personal care agent). Optionally, the amount of epigallocatechin gallate is at 30 wt. % (e.g., based on total wt. % of the personal care agent). Optionally, the personal care agent includes catechin, epicatechin, afzelechin, epiafzelechin, catechin gallate, epicatechin gallate, gallocatechin, epigallocatechin and rutin.

In certain embodiments, the natural extract is selected from cinnamon extract, black ginger extract, green tea extract, Emblica officinalis extract and a mixture of two or more thereof. In certain embodiments, the natural extract is selected from an extract of a Kaempferia species, a tea, pomegranate, sweet chestnut, camu camu, kakadu plum and Emblica officinalis. Optionally, the Kaempferia species is selected from black thai ginger, silver spot ginger, aromatic ginger and peacock ginger, preferably black thai ginger. Optionally, the tea is selected from green tea, black tea, oolong tea and white tea.

In certain embodiments, the personal care agent includes a natural extract selected from black thai ginger, green tea extract, trans-cinnamic acid, Emblica officinalis extract, and mixtures or blends thereof, and is absent of aluminum or aluminum-zirconium salts. Optionally, the personal care agent consists of consists of black thai ginger or green tea extract or a blend/mixture of black thai ginger and trans-cinnamic acid or a blend/mixture of green tea extract and Emblica officinalis extract.

In certain embodiments, the personal care agent includes from 1.5 wt. % to 10.0 wt. % based on the total weight of the personal care composition. Optionally, the personal care agent consists of 1.5 wt. % black thai ginger and 1.5 wt. % trans-cinnamic acid. Optionally, the personal care agent consists of 1.5 wt. % green tea extract and 1.5 wt. % Emblica officinalis extract.

In certain embodiments, the personal care agent further includes cyclomethicone, aluminum starch succinate, phenyl trimethicone, butyl ether, fumed silica, hydrogenated castor oil and stearyl alcohol.

In certain embodiments, the personal care agent exhibits a sweat reduction greater than 30% when compared to baseline.

In certain embodiments, the personal care agent does not include an aluminum chlorohydrate or aluminum zirconium tetrachlorohydrex gly. Optionally, the personal care composition does not include an aluminum-containing personal care agent, and optionally does not include aluminum and/or zirconium.

In certain embodiments, the personal care agent is one or more of an antioxidant, a protein coagulant, a preservative, and an anti-bacterial agent.

The personal care composition is effective to reduce or prevent perspiration, eliminate or mask body odor, and tone or reduce effects of skin aging. In certain embodiments, antiperspirant agents of the invention plug the pores of sweat glands by causing protein aggregation and/or by binding to specific receptors in the wall of the sweat duct. It has surprisingly been found that flavonoids have an antiperspirant effect. It is believed that these compounds cause coagulation or agglomeration of proteins. Typically, the antiperspirant agent and/or natural extract has at least a 20% sweat reduction in at least 50% subjects tested, as measured according to US FDA Guidelines (Reference 3). The term “subjects tested” is synonymous with the “target population” mentioned in the US FDA Guidelines.

### Personal Care Composition

The personal care composition of the invention comprises a personal care agent that is present as a natural extract or source as defined above. In certain embodiments, the personal care agent is derived/derivable or obtained/obtainable from the natural extract. Alternatively, the personal care agent is an aluminum-free synthetic/chemical material that demonstrates or imparts functional properties that are equivalent or substantially equivalent to the natural extract.

Typically, the personal care agent as defined above is the sole personal care agent in the personal care composition.

When the personal care composition comprises a natural extract as defined above, in certain embodiments, the natural extract is the sole personal care agent.

The personal care composition includes an effective amount of the personal care agent or the natural extract. The effective amount is an amount that will reduce or prevent perspiration, eliminate or mask body odor, tone or reduce the effects of skin aging, improve appearance, cleanse, or general aesthetics.

In certain embodiments, the personal care composition, e.g., antiperspirant, deodorant or astringent composition, includes a total amount of from about 0.1 to about 25.0 wt % of the personal care agent, e.g., antiperspirant, deodorant or astringent agent, or from about 0.5 to about 15.0 wt %, or from about 1.0 to about 10.0 wt %, such as from about 1.0 to about 5.0 wt % or from about 1.5 to about 10.0 wt. % of the antiperspirant agent.

In certain embodiments, the personal care composition, e.g., antiperspirant, deodorant or astringent composition, includes a total amount of from about 0.1 to about 4.0 wt % of the personal care agent, e.g., antiperspirant, deodorant or astringent agent, such as about 0.5 to about 4.0 wt %, or from about 1.0 to 4.0 wt %, such as about 1.0 to about 3.5 wt % or from about 1.5 to about 3.0 wt % of the antiperspirant agent.

In certain embodiments, the personal care composition, e.g., antiperspirant, deodorant or astringent composition, includes a total amount of from about 5.0 to about 40.0 wt % of the natural extract or material derived/derivable or obtained/obtainable therefrom or aluminum-free synthetic/chemical functional equivalent thereof, preferably from about 10.0 to about 30.0 wt %, more preferably from about 15.0 to about 25.0 wt % of the natural extract.

The amount of natural extract or material derived/derivable or obtained/obtainable therefrom or aluminum-free synthetic/chemical functional equivalent thereof, that is included in the personal care composition, e.g., antiperspirant, deodorant or astringent composition, depends on the composition of the extract, particularly the type and amount of ingredients that are present in the natural extract or agent obtained or obtainable therefrom.

In general, the personal care composition is a cosmetic composition, particularly a cosmetic composition for personal care.

The personal care composition is typically for topical use. Thus, in certain embodiments, the personal care composition is a topical personal care, e.g., antiperspirant, deodorant, astringent, shampoo, and body wash, composition.

The personal care composition is for topical application to skin, preferably a surface of the skin. In certain embodiments, the personal care composition is formulated for a specific mode of topical application.

Typically, the personal care composition comprises a topically acceptable carrier. The topically acceptable carrier depends on the type of formulation and its mode of topical application. In certain embodiments, the topically acceptable carrier is selected from water, an alcohol, an ether, an oil, a wax or combinations thereof. Optionally, the topically acceptable carrier is selected from water, oil, and combinations thereof, such as when the personal care composition is in the form of a gel, a paste, a liquid, a lotion or a foam. Optionally, the topically acceptable carrier is an alcohol. Suitable alcohols include but are not limited to mono-alcohol, polyhydric alcohol (e.g., an alcoholic compound having two or more hydroxy groups), and combinations thereof. When the alcohol is a mono-alcohol, the mono-alcohol is selected from ethanol and a fatty alcohol. Optionally, the fatty alcohol has a straight chain length of 6 to 18 carbon atoms. Optionally, the fatty alcohol is stearyl alcohol. When the alcohol is a polyhydric alcohol, the polyhydric alcohol is selected from polymeric alcohol and a monomeric alcohol. Optionally, the polymeric alcohol is a polyalkylene glycol, such as PEG or PPG. Optionally, the monomeric alcohol is selected from ethylene glycol, propylene glycol, glycerol or a sugar, such as glucose, sorbitol, xylitol, and combinations thereof. In certain embodiments, the topically acceptable carrier is an ether. Optionally, the ether is PPG-14 butyl ether. In certain embodiments, the topically acceptable carrier is an oil. Optionally, the oil is hydrogenated castor oil.

In certain embodiments, the personal care composition is provided as, or included in, a deodorant, such as a body deodorant for personal hygiene purposes.

In certain embodiments, the personal care composition is in the form of a cream, a gel, a paste, a liquid, a lotion, a foam, a scrub or a powder. Optionally, the personal care composition is a leave-on composition. Optionally, a leave on composition is applied to skin, or a surface of the skin, and left on for a period of time (e.g., from 1 minute to 24 hours), after which it is wiped or rinsed off with water, usually during the regular course of washing. When the personal care composition is in the form of a liquid, optionally, the liquid is a water-in-oil emulsion or an oil-in-water emulsion. In general, when the personal care composition is in the form of a liquid, optionally, the liquid may be a sprayable liquid.

In certain embodiments, the personal care composition is for delivery as an aerosol. Optionally, the topically acceptable carrier comprises a propellant.

In certain embodiments, the personal care composition comprises a lubricant. Optionally, the lubricant is, for example, a cyclomethicone, a dimethicone, a phenyltrimethicone or an ethyl methicone. Optionally, the lubricant is a cyclomethicone and/or phenyltrimethicone.

In certain embodiments, the personal care composition of the invention further comprises a natural oil. Optionally, the natural oil is included for skin care benefits. Optionally, the natural oil comprises an unsaturated fat, such as a polyunsaturated fat (e.g. a triglyceride derived from linoleic acid) and/or a monounsaturated fat (e.g. a triglyceride derived from oleic acid).

In certain embodiments, the personal care composition further comprises an antioxidant ingredient. Optionally, an antioxidant ingredient is included to chemically stabilize the personal care composition or any other natural ingredient that is present, such as a natural oil, particularly a natural oil comprising a polyunsaturated fat and/or a monounsaturated fat. Optionally, the antioxidant ingredient is selected from ascorbic acid, butyl hydroxy toluene, pentaerythrityl tetra-di-t-butyl hydroxyhydrocinnamate and tris-(tetramethylhydroxypiperidinol) citrate.

Optionally, the personal care composition includes a fragrance.

Optionally, the personal care composition includes a thickener. Optionally, the thickener is selected from fumed silica or a cellulose ether, such as hydroxypropyl cellulose, hydroxypropyl methyl cellulose, hydroxyethyl cellulose, carboxymethyl cellulose, cetyl hydroxyethyl cellulose, hydroxybutyl methyl cellulose, methyl hydroxyethyl cellulose or xanthan gum. Optionally, the thickener is fumed silica.

In certain embodiments, the personal care composition includes a surfactant. Optionally, the surfactant is an anionic surfactant, a cationic surfactant, a non-ionic surfactant or an amphoteric surfactant.

In certain embodiments, the personal care composition includes a skin care agent, such as an emollient, a humectant, a skin barrier promoter or a skin appearance modifier. Suitable emollients include stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate and myristyl myristate.

In certain embodiments, the personal care composition comprises a wide range of other optional components, such as a binder, a biological additive, a buffering agent, a colorant, a fragrance, a conditioner, an exfoliating agent, a pH adjuster, a preservative, a skin soothing agent or a skin healing agent.

In certain embodiments, the personal care composition comprises a preservative and optionally one or more components selected from a binder, a biological additive, a buffering agent, a colorant, a fragrance, a conditioner, an exfoliating agent, a pH adjuster, a skin soothing agent and a skin healing agent.

In certain embodiments, the personal care composition is a deodorant composition or an astringent composition or a shampoo composition or a body wash composition.

In certain embodiments, the personal care composition can be manufactured by conventional methods known in the art.

In certain embodiments, a method of preparing a personal care composition includes preparing a personal care agent by mixing a natural material, e.g., phytochemical or natural extract, or a material derived/derivable or obtained/obtainable therefrom, or an aluminum-free synthetic/chemical functional equivalent, with a cosmetic acceptable carrier.

Optionally, the method includes adding to the personal care composition one or more of a lubricant, fatty alcohol, starch and thickener. Optionally, the lubricant, fatty alcohol, and carrier are combined to form a mixture; the mixture is heated to a temperature effective to melt solids present and a thickener is added, dispersing the thickener. Optionally, a starch is then added, followed by addition of the phytochemical or natural extract, or a material derived/derivable or obtained/obtainable therefrom, or an aluminum-free synthetic/chemical functional equivalent.

### Methods and Uses

The method or uses of the invention are non-therapeutic.

The personal care composition of the invention is for use as a cosmetic.

The personal care composition or the personal care agent is applied to skin, preferably topically applied to skin.

The personal care composition or the personal care agent is applied to a surface of the skin.

In general, the skin is of a mammal, preferably a human.

Typically, the personal care composition or the personal care agent is applied to an area of skin where perspiration, odor or skin aging, occurs or an improved aesthetic effect or appearance is desired. For the antiperspirant, preferably, an area of the skin where there are sweat glands, such as apocrine sweat glands or eccrine sweat glands, preferably apocrine sweat glands.

Optionally, the area of skin is on a thigh (e.g. inner thigh), the groin, the back, the upper lip, an armpit, the forehead, the knee (e.g. the back of the knee), the neck (e.g. back of the neck), the buttock, the chest, the scalp, feet or the hand. In certain embodiments, the area of skin is an armpit.

The personal care composition or the personal care agent may be applied by a direct body contact applicator, such as a roll-on lotion or a solid stick product.

Alternatively, the personal care composition or the personal care agent is applied by a non-contact applicator, such as by spraying onto the skin. Optionally, the non-contact applicator may be an aerosol spray, a pump spray or a squeeze spray.

In certain embodiments, the personal care agent, the natural extract and/or the personal care composition are for use as a deodorant.

### EXAMPLES

The invention will now be illustrated by the following non-limiting examples.

### Examples 1 to 5

Formulation examples 1 to 5 shown in Table 1 were prepared by conventional methods.

TABLE 1

Formulation (F) No.

| Ingredient | 1 | 2 | 3 | 4 | 5 |
|---|---|---|---|---|---|
| Natural extract | E1 | E2 | E4 | E3 + E4 | E3 |
| Cyclomethicone | ✓ | ✓ | ✓ | ✓ | ✓ |
| Aluminum starch | ✓ | ✓ | ✓ | ✓ | ✓ |
| succinate | | | | | |
| Phenyltrimethicone | ✓ | ✓ | ✓ | ✓ | ✓ |
| PPG-14 butyl ether | ✓ | ✓ | ✓ | ✓ | ✓ |
| Fumed silica | ✓ | ✓ | ✓ | ✓ | ✓ |
| Hydrogenated | ✓ | ✓ | ✓ | ✓ | ✓ |
| castor oil | | | | | |
| Stearyl alcohol | ✓ | ✓ | ✓ | ✓ | ✓ |
| 3-phenoxyethanol- | ✓ | ✓ | ✓ | ✓ | ✓ |
| 1,2-propanediol | | | | | |

In Table 1 above, E1 is black thai ginger, E2 is ellagic acid, E3 is Emblica officinalis extract, and E4 is green tea extract. The extracts were obtained from Sabinsa Corporation. Each natural extract was included in an amount in each formulation to provide the same amount of antiperspirant agent across the formulations.

Formulation 1 included 3 wt. % black thai ginger, Formulation 2 included 3 wt. % ellagic acid, Formulation 3 included 3 wt. % green tea extract, Formulation 4 included 1.5 wt. % Emblica officinalis (saberry) extract and 1.5 wt. % green tea extract, and Formulation 5 included 3 wt. % Emblica officinalis (saberry) extract.

In addition, each of Formulations 1, 2, 3, 4 and 5 included 63.32 wt. % cyclomethicone, 7.66 wt. % phenyl trimethicone, 2.55 wt. % stearyl alcohol, 6.38 wt. % PPG-14 butyl ether, 3.83 wt. % hydrogenated castor oil, 3.83 wt. % fumed silica, and 8.93 wt. % aluminum starch octenylsuccinate.

### Comparative Examples

The formulations shown in Table 2 were prepared to provide a comparison formulations containing natural extracts. No natural extracts were included in these formulations.

TABLE 2

| | Comparative Formulation (CF) No. | |
|---|---|---|
| Ingredient | 1 | 2 |
| Natural extract | — | — |
| Antiperspirant | AP1 | AP2 |
| Cyclomethicone | ✓ | ✓ |
| Aluminum starch octenylsuccinate | ✓ | ✓ |
| Phenyltrimethicone | ✓ | ✓ |
| PPG-14 butyl ether | ✓ | ✓ |
| Fumed silica | ✓ | ✓ |
| Hydrogenated castor oil | ✓ | ✓ |
| Stearyl alcohol | ✓ | ✓ |

In Table 2 above, AP1 is aluminum chlorohydrate and AP2 is aluminum zirconium tetrachlorohydrate glycine (AZG).

The amount for each of cyclomethicone, aluminum starch octenylsuccinate, phenyltrimethicone, PPG-14 butyl ether, fumed silica, hydrogenated castor oil, and stearyl alcohol, was the same in both of the formulations (CF1 and CF2).

Formulation CF1 included 25.00 wt % antiperspirant and CF2 included 26.67 wt %.

In addition, Formulation CF1 included 49.35 wt. % cyclomethicone, 6.91 wt. % aluminum starch octenylsuccinate; 5.92 wt. % phenyl trimethicone, 4.93 wt. % PPG-14 butyl ether, 2.96 wt. % fumed silica, 2.96 wt. % hydrogenated castor oil, and 1.97 wt. % stearyl alcohol; Formulation CF2 included 48.26 wt. % cyclomethicone, 6.75 wt. % aluminum starch octenylsuccinate; 5.79 wt. % phenyl trimethicone, 4.82 wt. % PPG-14 butyl ether, 2.89 wt. % fumed silica, 2.89 wt. % hydrogenated castor oil, and 1.93 wt. % stearyl alcohol.

The method of preparing the formulations (1-5, CF1 and CF2) for a roll-on antiperspirant product is set out below.
1. Mix cyclomethicone, phenyl trimethicone, stearyl alcohol, PPG-14 butyl ether, and hydrogenated castor oil.
2. Heat to 82° C. to melt the solids.
3. While maintaining a temperature of 82° C., slowly add fumed silica using a high-speed disk type dispenser.
4. Once all silica is added, increase mixing speed so that peripheral velocity is almost 4000 ft/minute or higher for satisfactory dispersion.
5. Add aluminum starch octenylsuccinate followed by the Natural AP powder (i.e., for formulations 1-5) or the Antiperspirant powder (i.e., for the formulations CF1 and CF2). Each component is dispersed uniformly before adding the next.
7. Once all components are added, cool the mixture to 55° C.

### In Vivo Test Procedure

### Study Design

The formulation examples were tested in a standard hot room assessment clinical study. The study panellists underwent thermal stress after application of the antiperspirant compositions. The sweat reduction was determined by gravimetric collection of the sweat.
Subjects: 40 female panellists.
Conditions: 17-day washout period in which the panellists did not use any antiperspirants.
Test Design: Subjects underwent 5-day application period where each was given 400 mg±50 mg of one of the formulation examples. After the 5^{th} day, sweat collection was conducted after the panellists entered the hot room (100° F.±2° F. and 35%±5% relative humidity) for 80 minutes to induce sweating.

Analysis of Data: The data were transformed to their natural logarithms and the averages were analyzed according to the method described by Murphy and Levine (Reference 2). The analysis of covariance model included factors for sequence, subjects within a sequence, test articles, axilla and baseline as the covariate.

| Formulation | Timepoint | Mean | Mean Diff | % Change | p-value |
|---|---|---|---|---|---|
| 1 | Baseline | 0.64 | | | |
| | 8 Hours | 0.37 | −0.27 | −42.41% | 0.00 |
| 2 | Baseline | 0.63 | | | |
| | 8 Hours | 0.37 | −0.26 | −40.79% | 0.01 |
| 3 | Baseline | 0.73 | | | |
| | 8 Hours | 0.41 | −0.32 | −43.41% | 0.03 |
| 4 | Baseline | 0.71 | | | |
| | 8 Hours | 0.42 | −0.30 | −41.57 | 0.01 |
| 5 | Baseline | 0.54 | | | |
| | 8 Hours | 0.29 | −0.25 | −46.69 | 0.07 |

For each formulation, the % Change in sweat reduction was determined. The clinical study data showed that all of the formulations were effective at reducing sweat, with formulations F1 to F5 showing a sweat reduction greater than 30% when compared to baseline. A sweat reduction of at least 20% (as measured according to US FDA Guidelines) is considered to be suitable for commercial use as an antiperspirant agent.

The formulations F3 and F5 showed sweat efficacy results that are parity results when compared to the products with aluminum and aluminum zirconium.

### Examples 6 and 7

Formulation 6 included 1.5 wt. % black thai ginger and 1.5 wt. % of trans-cinnamic acid; Formulation 7 included 1.5 wt. % green tea extract and 1.5 wt. % Emblica officinalis (saberry).

In addition, each of Formulations 6 and 7 included 63.82 wt. % cyclomethicone, 7.66 wt. % phenyl trimethicone, 2.55 wt. % stearyl alcohol, 6.38 wt. % PPG-14 butyl ether, 3.83 wt. % hydrogenated castor oil, 3.83 wt. % fumed silica, and 8.93 wt. % aluminum starch octenylsuccinate.

The method of preparing the formulations 6 and 7 is set out below.
1. Mix cyclomethicone, phenyl trimethicone, stearyl alcohol, PPG-14 butyl ether, and hydrogenated castor oil.
2. Heat to 82° C. to melt the solids.
3. While maintaining a temperature of 82° C., slowly add fumed silica using a high-speed disk type dispenser.
4. Once all silica is added, increase mixing speed so that peripheral velocity is almost 4000 ft/minute or higher for satisfactory dispersion.
5. Add aluminum starch octenylsuccinate followed by the Natural powder (i.e., black thai ginger and trans-cinnamic acid for formulation 6 or green tea extract and Emblica officinalis for formulation 7). Each component is dispersed uniformly before adding the next.
7. Once all components are added, cool the mixture to 55° C.

### References

The following documents are referenced in the description.
1. A. P. Rauter et al.; “Nomenclature of flavonoids (IUPAC Recommendations 2017)”; Pure Appl. Chem., 90(9), (2018), 1429-1486.
2. T. D. Murphy & M. J. Levine; “Analysis of Antiperspirant Efficacy Test Results”; Journal of the Society of Cosmetic Chemists, 42, (1991), 167-197.
3. Guidelines for Effectiveness Testing of OTC Antiperspirant Drug Products”, published by the US FDA, when read in conjunction with § 350.60 (21 CFR 350.60) of the final monograph (final rule) for OTC antiperspirant drug products, published in the US FEDERAL REGISTER on Jun. 9, 2003 (68 FR 34273).

The entire disclosure of each document cited herein is hereby incorporated herein by reference. All references cited in this disclosure are incorporated by reference to the same extent as if each reference had been incorporated by reference in its entirety individually.

The citation and incorporation of patent documents herein is done for convenience only and does not reflect any view of the validity, patentability, and/or enforceability of such patent documents.

A number of embodiments of the disclosure have been described. Nevertheless, it will be understood that various modifications may be made without departing from the spirit and scope of the present disclosure. Accordingly, other embodiments are within the scope of the following claims.

All headings and sub-headings are used herein for convenience only and should not be construed as limiting the invention in any way.

We claim:

1. A personal care composition, comprising:
a personal care agent, comprising:
a phytochemical selected from a flavonoid, an ester of gallic acid, an ellagic acid, and a mixture of two or more thereof, or
a material derived/obtained from the phytochemical, or
an aluminum-free synthetic/chemical material that is an equivalent or imparts substantially equivalent functional properties as the phytochemical or the material derived/obtained from the phytochemical; and
a carrier.

2. The personal care composition according to claim 1, wherein the personal care agent comprises ellagic acid.

3. The personal care composition according to claim 1, wherein the personal care agent comprises a flavonoid.

4. The personal care composition according to claim 3, wherein the flavonoid is a 2-flavonoid.

5. The personal care composition according to claim 4, wherein the 2-flavonoid is a flavanone.

6. A personal care composition, comprising:
a personal care agent, comprising:
a first natural extract selected from black ginger extract, green tea extract, Emblica officinalis extract, and a mixture thereof, or
a second natural extract selected from an extract of Kaempferia species, a tea, pomegranate, sweet chestnut, camu camu, kakadu plum and Emblica officinalis, and a mixture thereof, or
a material derived or obtained from the first or second natural extract, or
an aluminum-free synthetic/chemical material that is an equivalent or imparts substantially equivalent functional properties as the first or second natural extract or the material derived or obtained from the natural extract; and
a carrier.

7. The personal care composition according to claim 6, wherein the personal care agent comprises from about 1.5 to about 10.0 wt. % based on total weight of the personal care composition.

8. The personal care composition according to claim 6, wherein the personal care agent comprises from about 1.5 to about 3 wt. % based on total weight of the personal care composition.

9. The personal care composition according to claim 6, wherein the black ginger extract comprises a flavan-4-one.

10. The personal care composition according to claim 9, wherein the flavan-4-one is selected from 5,7-dimethoxyflavan-4-one and 5-hydroxy-7-methoxyflavan-4-one.

11. The personal care composition according to claim 6, wherein the Emblica officinalis extract comprises glucogallin.

12. The personal care composition according to claim 6, wherein the natural extract is green tea extract is selected from catechin, epigallocatechin gallate and a combination thereof.

13. A method of preparing a personal care composition, comprising:
preparing a mixture, comprising:
a personal care agent, comprising:
a phytochemical selected from the group consisting of a flavonoid, an ester of gallic acid, ellagic acid or a mixture of two or more thereof, or
a natural extract selected from the group consisting of cinnamon extract, black ginger extract, green tea extract, Emblica officinalis extract, extract of a pomegranate, extract of a sweet chestnut, extract of a camu camu, extract of a kakadu plum, extract of extract of silver spot ginger, extract of aromatic ginger, extract of peacock ginger, and a mixture of thereof, or
a material derived/obtained from the phytochemical, or
a material derived/obtained from the natural extract, or
an aluminum-free synthetic/chemical materials that demonstrate or impart equivalent or substantially equivalent functional properties as the phytochemical, the natural extract, or the material derived/obtained from the phytochemical of the natural extract; and
a carrier.

14. The method of claim 13, wherein the mixture further comprises one or more materials selected from lubricant, fatty alcohol, thickener and starch.

15. A personal care composition, comprising:
a personal care agent, comprising:
a natural material selected from black thai ginger, green tea extract, trans-cinnamic acid, Emblica officinalis extract, and mixtures or blends thereof, and
absent of aluminum or aluminum-zirconium salts.

16. The personal care composition of claim 15, wherein the personal care agent is a mixture or blend of black thai ginger and trans-cinnamic acid.

17. The personal care composition of claim 15, wherein the personal care agent is a mixture or blend of green tea extract and Emblica officinalis extract.

18. The personal care composition of claim 15, wherein said composition exhibits a sweat reduction greater than 30% when compared to baseline.

19. The personal care composition of claim 15, wherein the personal care agent is a mixture or blend of about 1.5 wt. % black thai ginger and about 1.5 wt. % trans-cinnamic acid.

20. The personal care composition of claim 15, wherein the personal care agent is a mixture or blend of about 1.5 wt. % green tea extract and about 1.5 wt. % Emblica officinalis extract.
